(19) United States
(12) Patent Application Publication
(10) Pub. No.: US 2017/0231496 A1
(43) Pub. Date: Aug. 17, 2017

(54) WIRELESS BLOOD PRESSURE MEASURING TECHNIQUES

(71) Applicant: Carematix, Inc., Chicago, IL (US)

(72) Inventor: Sukhwant Singh Khanuja, Chicago, IL (US)

(21) Appl. No.: 15/460,873

(22) Filed: Mar. 16, 2017

Related U.S. Application Data
(63) Continuation of application No. 14/099,128, filed on Dec. 6, 2013, now abandoned.
(60) Provisional application No. 61/734,021, filed on Dec. 6, 2012.

(51) Int. Cl. A61B 5/00; A61B 5/021

(52) U.S. Cl. CPC A61B 5/0022; A61B 5/021; A61B 5/746; A61B 5/742

(57) ABSTRACT

According to certain inventive techniques, a blood pressure recording device includes at least one blood pressure sensor, a display configured to visually present received data, a wireless transceiver, a memory storing a reminder time, and a processor including a clock. The processor is configured to: receive blood pressure data from the at least one blood pressure sensor; process the blood pressure data to form a systolic reading and a diastolic reading; transmit the systolic reading and the diastolic reading to the display for visual presentation to a user; transmit the systolic reading and the diastolic reading to the wireless transceiver for wireless transmission; retrieve the reminder time from the memory; compare the reminder time to a time of the clock; and generate an alert when the clock time is equal to the reminder time.

### CROSS REFERENCE TO RELATED APPLICATIONS

This application is a continuation of U.S. patent application Ser. No. 14/099,128 filed on Dec. 6, 2013, which claims priority to U.S. Provisional Patent Application No. 61/734,021 filed on Dec. 6, 2012, the entirety of these applications is herein incorporated by reference.

### FEDERALLY SPONSORED RESEARCH OR DEVELOPMENT

[Not Applicable]

### JOINT RESEARCH AGREEMENT

[Not Applicable]

### SEQUENCE LISTING

[Not Applicable]

### BACKGROUND

The present invention generally relates to medical devices and particularly relates to vital sign monitoring medical devices such as blood pressure recording devices. The present invention more particularly relates to techniques for recording and storing blood pressure of an individual at a data processing system through at least one wireless link (for example, a Wi-Fi or cellular link).

Blood pressure monitors include manual blood pressure monitors and digital blood pressure monitors. Manual blood pressure monitors may include an arm cuff, a squeeze bulb, and a gauge to measure blood pressure. Digital blood pressure monitors may be available with wrist cuffs and/or arm cuffs. These monitors may have a display unit that displays the results.

One type of known medical monitoring device employs a remote transmission function to send measured medical data to a remote user. This medical monitoring device allows a health service provider or patient's relative at a remote distance to observe the medical data of the patient being measured at home through the Internet.

Another type of known medical monitoring device is blood pressure monitor with a wireless transmitter to send measured physiological data from a pressure sensor to a remote device having a wireless receiver. The blood pressure recording system for use with this monitor employs a portable device such as a mobile phone, wherein a software program is installed on the portable device to process the received data. The remote device can forward the data to other remote devices.

Another known technique employs a portal to facilitate collection and storage measured medical monitoring data acquired from a vital sign monitoring devices in a central server.

### SUMMARY

Certain inventive techniques may provide a new and improved system for recording blood pressure and in particular the systolic, diastolic and mean blood pressures with a time stamp of when the measurement is taken.

Certain inventive techniques may also provide a method and system for storing the recorded blood pressure in a data processing system through a wireless transceiver.

Certain inventive techniques may also provide a method that can be applied for other medical devices like ECG machines or pulse monitors for recording physiological data and storing recorded data in a data processing system through a wireless (for example, Wi-Fi or cellular) transceiver.

Certain inventive techniques may also provide a portal wherein the user or the family doctor can log in with an assigned identification number and review the stored blood pressure data in the data processing system.

Certain inventive techniques may also provide a system and method for retrieving the measured and stored blood pressure details as needed.

Certain inventive techniques may also provide reminders to users to complete tasks like take weight reading as preconfigured on the web portal by using illuminated buttons on the blood pressure recording device, audible beeps and using the display screen to display a message.

According to certain inventive techniques, a method includes receiving, at a data processing system, a request to set a reminder time for a blood pressure recording device. At the data processing system, the reminder time is associated with a user and stored in association with other user data. The reminder time is transferred to the blood pressure recording device via a first transmission path, wherein at least a portion of the first transmission path includes a wireless segment (for example, a Wi-Fi segment). The blood pressure recording device receives and stores the reminder time in a memory. The reminder time is compared to the clock time. If the clock time is equal to the reminder time, an alert is generated (for example, an audible or visible alert).

The method may further include generating, by the blood pressure recording device, blood pressure data (for example, systolic data, diastolic data, or a time stamp corresponding to the time at which the blood pressure data was generated) for the user. Blood pressure data may include related types of data, such as pulse rate or pulse mark. The blood pressure data may be transmitted to the data processing system via a second transmission path, wherein at least a portion of the second transmission path includes a wireless segment (for example, a Wi-Fi segment). The data processing system may receive the blood pressure data and associate it with the user. The data processing system may store the blood pressure data in association with other user data.

According to certain inventive techniques, a blood pressure recording device includes at least one blood pressure sensor, a display configured to visually present received data, a wireless transceiver, a memory storing a reminder time, and a processor including a clock. The processor is configured to: receive blood pressure data from the at least one blood pressure sensor; process the blood pressure data to form a systolic reading and a diastolic reading; transmit the systolic reading and the diastolic reading to the display for visual presentation to a user; transmit the systolic reading and the diastolic reading to the wireless transceiver for wireless transmission; retrieve the reminder time from the memory; compare the reminder time to a time of the clock; and generate an alert when the clock time is equal to the reminder time.

The blood pressure recording device may also have a speaker and the processor may be configured to generate the alert by causing the speaker to generate an audible alert. The blood pressure recording device may also have a lamp and the processor may be configured to generate the alert by causing the lamp to generate a visible alert.

The processor of the device may be further configured to: associate a time stamp with the blood pressure data, wherein the time stamp corresponds to when the blood pressure data is received from the at least one blood pressure sensor; and transmit the time stamp along with the systolic reading and the diastolic reading to the wireless transceiver for wireless transmission.

According to certain inventive techniques, a blood pressure recording device includes: at least one blood pressure sensor; a display configured to visually present received data; a wireless transceiver; a memory storing a reminder time; and a processor including a clock, and configured to: receive blood pressure data from the at least one blood pressure sensor; process the blood pressure data to form a systolic reading and a diastolic reading; transmit the systolic reading and the diastolic reading to the display for visual presentation to a user; transmit the systolic reading and the diastolic reading to the wireless transceiver for wireless transmission; retrieve the reminder time from the memory; determine a window of time based on the reminder time and window time data; determine whether blood pressure data has been received during the window of time; and if blood pressure data has not been received during the window of time, generate an alert.

The blood pressure recording device may also include a speaker, and wherein the processor is further configured to generate the alert by causing the speaker to generate an audible alert. The blood pressure recording device may also include a lamp, and wherein the processor is further configured to generate the alert by causing the lamp to generate a visible alert. The processor may further be configured to: associate a time stamp with the blood pressure data, wherein the time stamp corresponds to when the blood pressure data is received from the at least one blood pressure sensor; and transmit the time stamp along with the systolic reading and the diastolic reading to the wireless transceiver for wireless transmission.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF SEVERAL VIEWS OF THE DRAWINGS

FIG. 1 is a block diagram of a system for measuring and storing vital sign parameters from a portable vital sign monitoring device connected to patient, according to certain inventive techniques.

FIG. 2 depicts a wireless-enabled blood pressure recording device, according to certain inventive techniques.

FIG. 3 illustrates a display unit of the blood pressure recording device, according to certain inventive techniques.

FIG. 4 depicts a display unit of the blood pressure recording device in an access point mode, according to certain inventive techniques.

FIG. 5 depicts a display unit of the blood pressure recording device illustrating error indications, according to certain inventive techniques.

FIG. 6 illustrates a graphical user interface for displaying data and for configuring a blood pressure recording device, according to certain inventive techniques.

FIG. 7 is a flow chart illustrating a method for measuring and storing vital sign parameters acquired from a portable vital sign monitoring device connected to a patient, according to certain inventive techniques.

FIG. 8 illustrates a system for recording blood pressure of a user, according to certain inventive techniques.

FIGS. 9A and 9B illustrate flow charts for methods of recording blood pressure of a user, according to certain inventive techniques.

The foregoing summary, as well as the following detailed description of certain techniques of the present application, will be better understood when read in conjunction with the appended drawings. For the purposes of illustration, certain techniques are shown in the drawings. It should be understood, however, that the claims are not limited to the arrangements and instrumentality shown in the attached drawings. Although the specific features of the present invention are shown in some drawings and not in others. This is done for convenience only as each feature may be combined with any or all of the other features in accordance with the present invention.

### DETAILED DESCRIPTION

In the following detailed description, reference is made to the accompanying drawings that form a part hereof, and in which the specific inventive techniques that may be practiced are shown by way of illustration. These techniques are described in sufficient detail to enable those skilled in the art to practice the embodiments and it is to be understood that the logical, mechanical and other changes may be made without departing from the scope of the inventive techniques. The following detailed description is therefore not to be taken in a limiting sense.

Various inventive techniques provide a method and system for a wireless-enabled blood pressure recording device. According to one technique of the present invention, the system for recording blood pressure of an user and storing the recorded data in the data processing system may include a blood pressure recording device for recording a patient's blood pressure, a data processing system for providing storage and data review facility, a wireless transceiver embedded in the blood pressure recording device for transmitting the recorded data to the data processing system, and a portal for facilitating interaction between a user and the data processing system. The recorded blood pressure data may include a systolic pressure value, a diastolic pressure value, a heart rate, and a date/time value. The blood pressure recording device may record the blood pressure using an arm cuff which may be wrapped around a person's upper arm.

According to one inventive technique, the blood pressure recording device may include a blood pressure recording means for recording blood pressure, a processor for processing the recorded blood pressure, a display unit for displaying the processed recorded blood pressure, a storage unit for storing the recorded blood pressure data, an internal battery for powering the blood pressure recording device, one or more multifunction configurable buttons, or an external battery for powering the wireless transceiver embedded in the blood pressure recording device. The display unit of the blood pressure recording device may be a Liquid Crystal Display (LCD) screen. The display unit may display a systolic reading value, a diastolic reading value, a pulse rate, a low battery indication, a User1 indication, a User2 indication, error indication(s), an access point mode, or communication status with the data processing system or wireless router.

According to one inventive technique, the blood pressure recording device may include a wireless transceiver to transmit the blood pressure data to the data processing system. The blood pressure recording device may be equipped with a wireless network transceiver to pick up signals transmitted from a wireless access point or router to enable the blood pressure recording device to connect to the data processing system through a wireless communication and over the Internet.

According to one inventive technique, a user may interact with the data processing system via a portal. The user may first register with the data processing system via the portal by providing credentials like name, address, age, blood pressure recording device serial number, user number, or the like. The portal in turn may display a unique identification indicator for the user. The blood pressure recording device may be connected to the user's body and the blood pressure of the user may be recorded. The recorded blood pressure may be displayed on the display unit of the blood pressure recording device. The displayed blood pressure may include a systolic pressure value and a diastolic pressure value. The recorded blood pressure data (potentially including the systolic blood pressure, diastolic blood pressure, heart rate, time stamp, or unique identification number) is stored in the storage unit. The wireless transceiver may transmit the blood pressure data from the blood pressure recording device to the router point. The router may transmit the data to the data processing system via, for example, the Internet.

According to one inventive technique, the user may log into the data processing system via the portal by entering the assigned identification number or login credentials and may retrieve the blood pressure data.

According to one inventive technique, the blood pressure data stored in the data processing system can either be shared with the family doctor or the patient can retrieve the stored blood pressure data, whenever needed by entering appropriate credentials into the portal.

According to one inventive technique, the system of the present invention may be designed in such a way that a system comprises multiple medical monitoring devices interfaced with one another to measure the respective physiological data. For example, a single system may include a blood pressure recording device and an ECG monitoring device so that the user is able to measure both the physiological data and the measured data are stored in the data processing system.

According to one inventive technique, the present invention is not limited to measure the blood pressure using a blood pressure recording device. Certain inventive techniques may also be designed in such a way that the wireless transceiver may be attached to other medical monitoring devices and corresponding physiological data may be measured and stored in the data processing system.

According to one inventive technique, the blood pressure recording device may further comprise a transceiver to connect to a wireless network. The recorded data from the blood pressure recording device may then be sent to the data processing system using the wireless network.

According to one inventive technique, the blood pressure recording device may provide reminder alerts to a user. The alerts may be in the form of notifications that indicate a user about an “unfinished” or “to be done” task. The reminder may be set in the data processing system by a user via the portal. A connection may be established between the data processing system and the blood pressure recording device (for example, via a wireless router). Once a desired reminder is set in the data processing system, the data processing system may forward the reminder details to the blood pressure recording device when a connection is established between the data processing system and the blood pressure recording device. The data processing system may update the blood pressure recording device with the reminder details set by the user. The blood pressure recording device may provide reminders relating to a task, for example, for measuring blood pressure data. The reminder may be in the form of audible alarm, illumination of buttons, and/or on the LCD display, for example.

According to one inventive technique, the system of the present invention may be a portable device. The device may be designed in such a way that it is either used at home or hospitals, for example. Also the recording device may be easily operated by a normal user or even by a semi-skilled person.

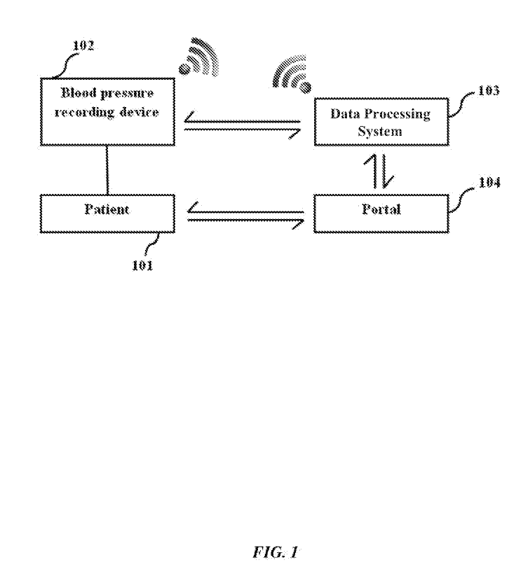

FIG. 1 is a block diagram of a system for measuring and storing vital sign parameters (for example, blood pressure parameters) from a portable vital sign monitoring device connected to patient, according to certain inventive techniques. With respect to FIG. 1, the patient 101 is registered with a data processing system 103 via a portal 104 by providing credentials like name, address, age, blood pressure unit serial number, user number, or the like. Once the credentials are filled in the data processing system 103, data processing system 103 assigns a unique identification number and login credentials to the patient 101. The arm cuff of the blood pressure recording device 102 is wrapped on the patient's upper arm and the blood pressure is measured. The blood pressure recording device 101 comprises a wireless antenna (for example, Wi-Fi) adopted within the blood pressure recording device 102 for providing a wireless connectivity. The radio signals are transmitted to the router from the wireless antenna. These radio signals are received and decoded by the router which is adopted within an access point. The access point transmits the information received by the radio signals to the data processing system 103 using a modem. This modem may connect to the data processing system 103 via phone line, broadband or cellular networks. The data processing system 103 stores the blood pressure data and supplies it to individual user whenever needed. The wireless transceiver connection is bi-directional. In the reverse process, the broadband modem sends the information to the router adopted within the access point. The router then translates the signals and transmits to the wireless enabled blood pressure recording device 102 for viewing by a user.

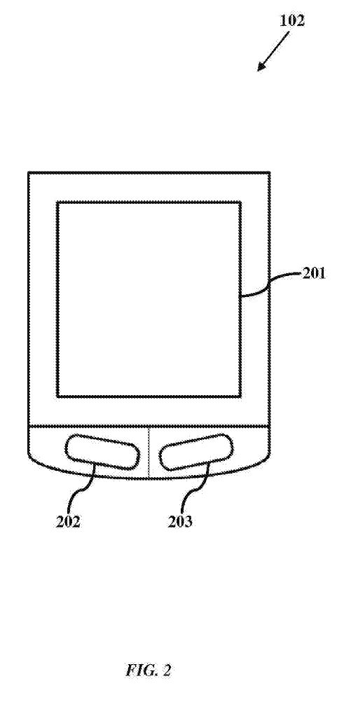

FIG. 2 is a wireless enabled blood pressure recording device 102, according to certain inventive techniques. The wireless enabled blood pressure recording device 102 of the present invention may include a blood pressure recording means for recording blood pressure, a processor for processing the recorded blood pressure, a display unit 201 for displaying the processed recorded blood pressure, a storage unit (memory) for storing the recorded blood pressure data, an internal battery for powering the blood pressure recording device, one or more multifunction configurable buttons (202 and 203), and an external battery for powering a wireless transceiver embedded in the blood pressure recording device. The recorded blood pressure data may be stored in a data processing system. The recorded blood pressure data may include a systolic pressure value and a diastolic pressure value. The user may be connected to the data processing system via a portal.

The user may register with the data processing system via the portal by providing the user credentials like name, address, age, blood pressure recording device serial number, user number, or the like. The data processing system in turn may assign a unique identification number for the user. The data processing system may store the user credentials and the blood pressure data corresponding to the blood pressure recording device 102. The data processing system may check for any new stored data in the blood pressure recording device 102 from the last updating date. When new blood pressure recorded data stored in the blood pressure recording device 102 is detected, the same may be updated in the data processing system. The data processing system may stores the recorded blood pressure data of the user. Also there may be a time clock in the blood pressure recording device 102 which may be synchronized with the data processing system when the blood pressure recording device 102 communicates with the data processing system. The blood pressure recording device 102 may time stamp data using this time clock when the blood pressure of the user is recorded.

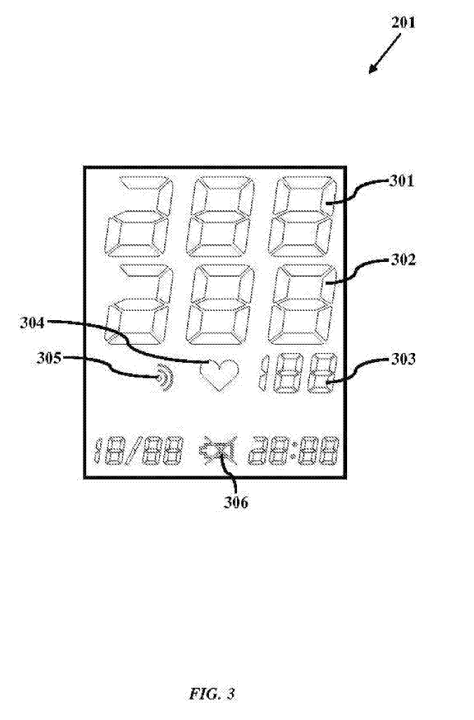

FIG. 3 illustrates the display unit 201 of the blood pressure recording device, according to certain inventive techniques. The display unit 201 of the blood pressure recording device may display systolic pressure 301, diastolic pressure 302, pulse rate per minute 303, pulse mark 304, data transmission 305, and low battery indicator 306. The display unit 201 may also display error indications and access point (“AP”) mode (not shown in FIG. 3). The systolic reading value and the diastolic reading value may be displayed in mmHg. The pulse rate may be displayed as beats per minute. The low battery status may be indicated by displaying low battery icon on the display unit 201 and the processor of the blood pressure recording device may send the low battery indication to the wireless transceiver.

According to certain inventive techniques, when User 1 (first user) button is pressed, the “U1” indication is displayed on the display unit. Similarly, when User 2 (second user) button is pressed, the “U2” indication is displayed on the display unit.

According to certain inventive techniques, when the processor receives a message from the wireless transceiver that the communication with data processing system has started, then the display may blink the data transmission icon 305 on the display unit. The blink rate of the data transmission icon 305 may be 1 second ON, 2 second OFF. Similarly, when the processor receives a message from the wireless transceiver that the wireless transceiver is communicating with the data processing system, then the display stops blinking of the data transmission icon 305 and leaves the data transmission icon 305 in an “ON” state on the display unit. Similarly, when the processor receives a message from the wireless transceiver that the communication with data processing system has ended, then the display may turn OFF the data transmission icon 305.

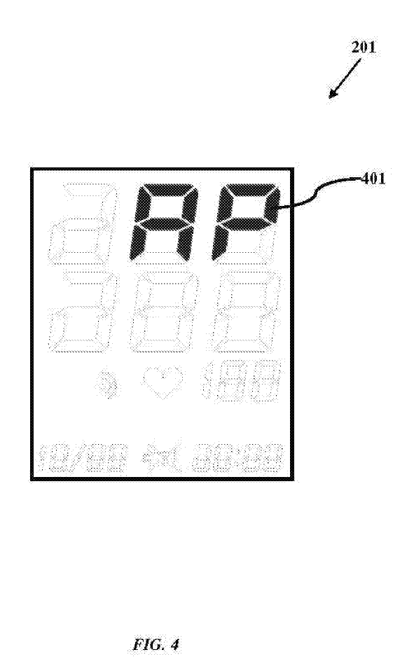

According to certain inventive techniques, when the processor receives a message from the wireless transceiver that the communication with data processing system has failed, then the display displays an error indications “E3” on the display unit 201 (See, for example, FIG. 4).

According to certain inventive techniques, when the external battery is inserted in to the blood pressure recording device, the processor may display Access Point Mode “AP” on the display unit for a predetermined time period (for example, the “AP” may be displayed for 2 minutes).

According to certain inventive techniques, the user is allowed to select any one multifunction configurable buttons at one time. When multifunction configurable button is selected, the blood pressure recording device switches ON and displays the multifunction configurable button or user flags U1 and U2 selected in the display unit 201. A reminder can be set to display an alert message in the display unit 201 with respect to the user selected user flags U1 and U2 after a predetermined time. The predetermined times may be set via the portal by the user or an authorized person. These time settings may be sent to the blood pressure recording device when the blood pressure recording device communicates with the data processing system.

According to certain inventive techniques, upon pressing of the User 1 or User 2 button at once, the blood pressure recording device may start inflating the cuff and begin to record blood pressure. If the user presses the button (either of User 1 button or User 2 button) again, the inflating of the cuff may stop (blood pressure recording is stopped) and the cuff may be deflated.

According to certain inventive techniques, each of the user buttons tracks a separate group of data. The User 1 or User 2 buttons can be used by two users individually, or one person can track their blood pressure at home and work, during the day and night, or the like.

According to certain inventive techniques, the wireless transceiver may be connected to the blood pressure recording device to collect the data or to connect to the access point to transmit the data. The blood pressure recording device may be switched ON by pressing either of the multifunction configurable buttons. The selected multifunction configurable button may be indicated in the display unit 201 by the user flag indicator U1. A processor of the blood pressure recording device may process the recorded blood pressure and cause display of the recorded data and store the data in the storage unit. An external battery is optional. When the user wishes to store the recorded blood pressure data in the data processing system, an external battery may be inserted into a slot provided in the blood pressure recording device. The external battery may power the wireless transceiver and the wireless transceiver's indicator (data transmission indicator) 305 may be displayed in the display unit 201 of the blood pressure recording device. If the external battery is removed, the blood pressure recording device may function normally, except powering the wireless transceiver. The wireless transceiver's connection may be bi-directional. The blood pressure recording device may be connected to the data processing system via wireless transceiver for storing the recorded blood pressure data and also retrieving the configurations or preferences set by the user, from the data processing system to the blood pressure recording device. The user can login to the data processing system via the portal by entering the assigned identification number and retrieve the blood pressure data from the data processing system as needed.

FIG. 4 illustrates a display unit 201 of the blood pressure recording device illustrating wireless transceiver in Access Point mode, according to certain inventive techniques. The blood pressure recording device may be switched ON by pressing either of the multifunction configurable buttons. The blood pressure recording device may display the user selected multifunction configurable button in the display unit 201. The Access Point mode may comprise switching ON the wireless transceiver of the blood pressure recording device by inserting an external battery into a slot provided in the blood pressure recording device. The activation of the Access Point mode may be indicated by illuminating an Access Point mode indicator 401 in the display unit 201 as shown in FIG. 4. Upon activation of the Access Point mode 401, the signals from the wireless transceiver of the blood pressure recording device may transmit a unique SSID of the blood pressure recording device wirelessly. The user may connect to the blood pressure recording device over Wi-Fi using another device like a smart phone or laptop, for example. The user may then open a portal and configure the blood pressure recording device with router settings required to connect and upload blood pressure data to the data processing system.

As a result, a wireless connection may be established between the blood pressure recording device and the local Access Point which is connected to the data processing system. The data processing system may check for any newly recorded blood pressure data in the blood pressure recording device from the last updating time. When any newly recorded blood pressure data is detected, the same may be updated in the data processing system. The data processing system may store recorded blood pressure data of the user. The user may login to the data processing system via the portal by entering the assigned identification number and retrieve the blood pressure data from the data processing system. A reminder can be set to display an alert message in the display unit 201 with respect to the multifunction configuration buttons after a predetermined time. The predetermined times may be set in the portal by the user or an authorized person. These time settings may be sent to the blood pressure recording device whenever the blood pressure recording device communicates with the data processing system.

A time clock may be maintained in the blood pressure recording device using an internal battery. This clock may be synchronized with the data processing system clock whenever the blood pressure recording device connects to the data processing system.

A user can set reminders via the portal in the data processing system in their account to remind to complete a task like taking a blood pressure reading or otherwise. Whenever the blood pressure recording device communicates with the data processing system it may download and store this reminder. At the reminder time, the respective user button may turn ON and the display may show the user number (for example, U1), and there may be an audible sound to remind the user to complete the task.

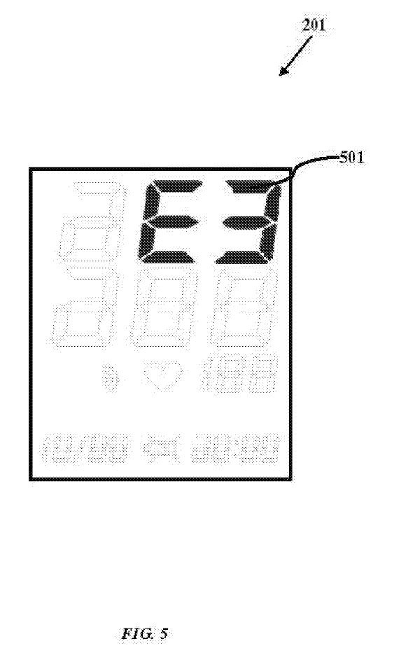

FIG. 5 is a display unit of the blood pressure recording device illustrating error indications, according to certain inventive techniques. When the processor of the blood pressure recording device receives a message from the wireless transceiver that the communication with data processing system has failed, then the processor may display an error indication such as “E3” 501 on the display unit 201 of the blood pressure recording device as shown in FIG. 5. The error indications may include to “EE” for low voltage or interior circuit problem, “E1” for abnormal situation during inflation, “E2” for artificial interference and noise or abnormal measurement or over safety pressure, “E3” for data processing system communication failure 501, and “E4” radio communication failure (measuring data failed wireless transmission).

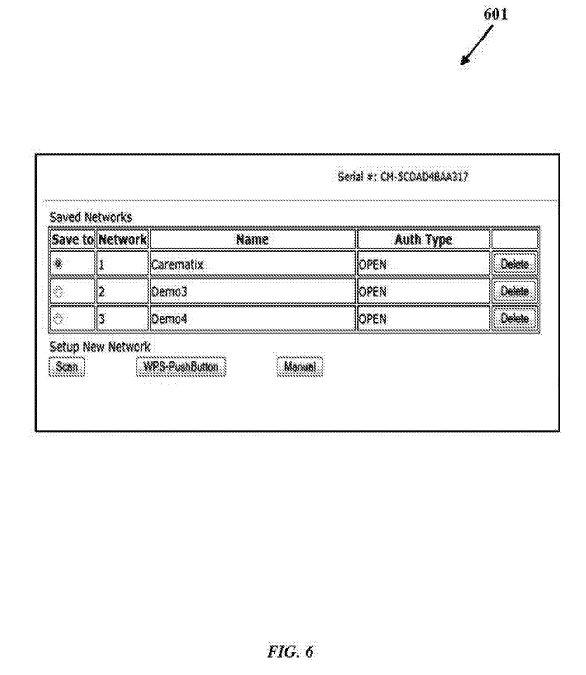

FIG. 6 illustrates a graphical user interface for configuring a blood pressure recording device, according to one embodiment of the present invention. The configuration page 601 in the portal for configuring the blood pressure recording device may comprise a list of saved networks (if there are any). Under the saved networks box, buttons (for example, Scan, WPS-Push Button, and Manual) may be provided as shown in FIG. 6. The buttons may be used for setting a new network connection. Upon selecting the Scan button, lists of active wireless networks may be displayed. The user may select at least one network, type the password into the “credential” field next to the network's name, and/or press “connect” to connect to the preferred network. If the blood pressure recording device connects to the network a “Successfully Connected” message may be displayed. If a connection is not established, an error message such as “Check the Password and Try Again, or try one of the other connection methods” may be displayed.

If the wireless router contains a blue WPS-Pushbutton, then the method for setting a new network may include the steps of selecting a WPS-button on the Wi-Fi router and selecting WPS-Pushbutton on the configuration page 601. If the blood pressure recording device connects to the network then a message may be displayed indicating “Successfully Connected.”

If the user has the SSID, WEP Key, or WPA password for a particular network, then the method for setting a new network may include selecting a “Manual” button on the configuration page 601 and entering the details manually. If the blood pressure recording device connects to the network then a message may be displayed indicating “Successfully Connected”.

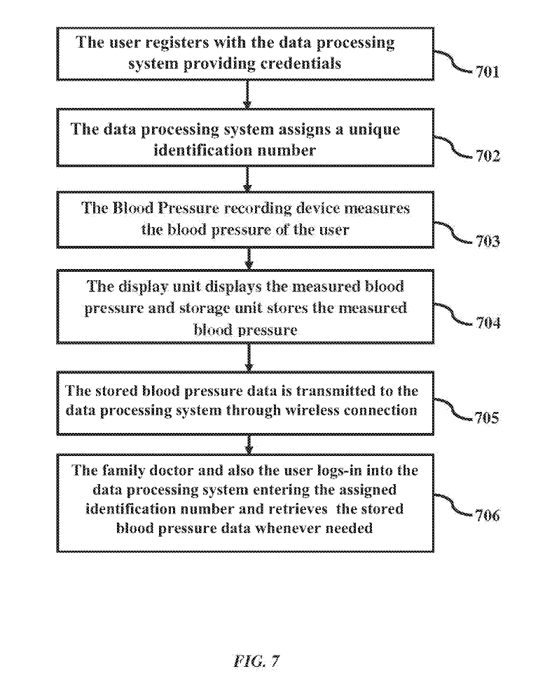

FIG. 7 is a flow chart illustrating a method for measuring and storing vital sign parameters acquired from a portable vital sign monitoring device connected to a patient, according certain inventive techniques. With respect to FIG. 7, the user may register with a data processing system via a portal by providing user credentials like name, address, age, blood pressure unit serial number, user number, or the like (701). Once the user credentials are filled in the portal, the data processing system may assign a unique identification number to the user (702). The arm cuff of the blood pressure recording device may be wrapped on the user's upper arm and the blood pressure may be measured (703). The display unit within the blood pressure recording device may display the blood pressure (for example, the systolic and diastolic pressure) and the storage unit within the recording device may store the measured blood pressure (704). The stored blood pressure may be transmitted to the data processing system by the blood pressure recording device through the wireless transceiver connection (705). The family doctor or the user himself may be able to login to the portal by entering the assigned identification number and retrieving the data (706).

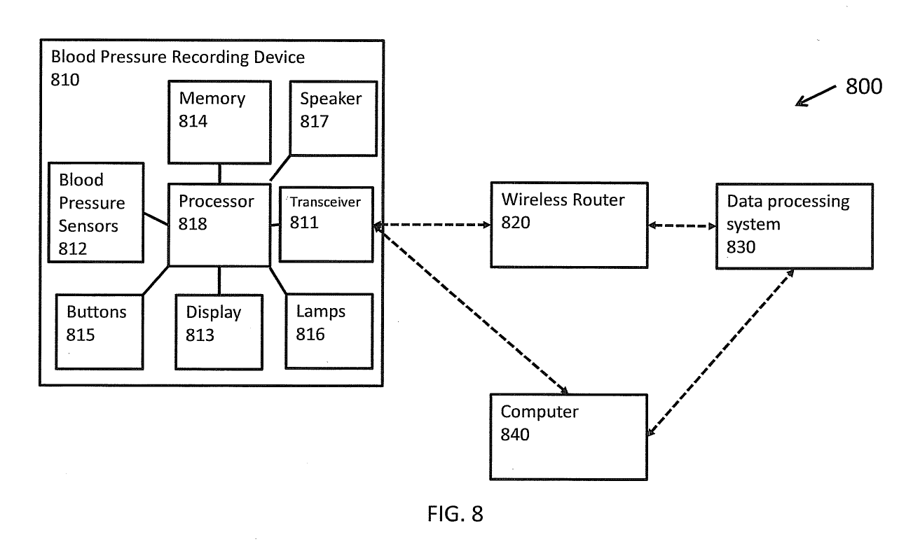

FIG. 8 illustrates a blood pressure recording system 800, according to certain inventive techniques. The system 800 may include a blood pressure recording device 810, a wireless router 820, a data processing system, 830, and a computer 840. The device 810 may communicate wirelessly with the router 820, which in turn may communicate with the data processing system 830 via a network such as the Internet. Thus, the device 810 may communicate with the data processing system 830 via the router 820. A computer 840 may communicate with the data processing system 830 through a portal via a network such as the Internet. The computer 840 may also wirelessly communicate with the device 810.

The device 810 may include a wireless transceiver 811, blood pressure sensors 812, a display 813, memory 814, buttons 815, one or more lamps 816, a speaker 817, and a processor 818. The transceiver 811, display 813, speaker 817, and lamps 816 may be controlled by the processor 818. The blood pressure sensors 812, buttons, 815, and transceiver 811 may provide information to the processor 818.

The transceiver 811 may wirelessly transmit and receive signals to/from the router 820 and/or computer 840. The transceiver 811 may include a Wi-Fi transceiver (for example, a transceiver that is compliant with the Institute of Electrical and Electronics Engineers' 802.11 standards). The transceiver 811 may also include a cellular transceiver. The processor 818 may transmit data to the transceiver 811, which in turn may transmit the data to the router 820 or computer 840. The transceiver 811 may also communicate wirelessly received data to the processor 818. The transceiver 811 may communicate other information with the processor 818, such as whether a wireless link exists, the identity of wireless nodes and networks, error messages, or the like.

In response to the user taking his or her blood pressure, the blood pressure sensors 812 may generate one or more signals that are communicated from the sensors 812 to the processor 818 where it is received. The blood pressure signals may be used to determine blood pressure and/or related data of the user on the device 810.

The display 813 may be an LED, LCD, or other type of display. The display 813 may be backlit. The display 813 may receive display information from the processor 818 and responsively form a visual arrangement to communicate the display information to a user.

The memory 814 may be a volatile or non-volatile memory and may communicate with the processor 818. The memory 814 may store various types of information disclosed herein. The memory 814 may be integrated with the processor 818, for example, in a chip. The memory 814 may include one or more memories. Similarly, the processor 818 may include one or more processors.

Buttons 815 may include buttons that can be actuated by a user. Some of the buttons 815 may be configurable and/or may have multiple functions. For example, actuation of some buttons 815 may cause the processor 818 to perform a user-configurable activity. Such activity may be configurable through the computer 840 or data processing system 830. The computer 840 may program the device 810 to perform a configurable activity either through direct communication with the device 810 or communication through the data processing system 830 and the router 820. Configurable parameters discussed above may be stored in the memory 814. Other buttons may include a power button, a transceiver enable/disable button, or an illumination button.

Lamps 816 may cause illumination of various parts of the device 810. For example, one lamp 816 may illuminate the display 813 (from the front or back). Other lamps 816 may illuminate buttons 815. For example, a lamp 816 may provide visual feedback to a user to indicate that a button 815 has been pressed. A lamp 816 may be used as a reminder to the user to take action, as will be further discussed below. A lamp 816 may include one or more LEDs or other light-emitting devices.

A speaker 817 may generate sounds. For example, the speaker 817 may make beeps or other sounds to alert a user to a given condition (for example, low battery or loss of network connectivity) or alarm time, as will be discussed in more detail below.

According to certain inventive techniques, the processor 818 may receive blood pressure signal(s) from the sensors 812. From the blood pressure signal(s), the processor 818 may determine blood pressure data that corresponds to the blood pressure of a user. The processor 818 may transmit the blood pressure data to the transceiver 811. The processor 818 may also transmit the blood pressure data to the display 813. The processor 818 may receive a reminder time from the memory 814. The processor 818 may compare the reminder time to a time of a clock. If the time of the clock is equal to the reminder time, an alert may be generated. For example, the processor 818 may cause the speaker 817 to emit a sound or may cause the lamps 816 or display 813 to illuminate.

A reminder time may include or be associated with a window of time. The processor 818 may only cause an alert to be generated if the time of the clock exceeds that of the reminder time plus the window, and only if no blood pressure reading has been taken. For example, say the reminder time is 9:00 AM and a window of time is 3 hours. The processor 818 may only cause an alert to be generated if the user does not use the blood pressure recording device 810 take his or her blood pressure within the reminder time plus the window of time (that is, between 9:00 AM and 12:00 PM). The window may also extend to times prior to the reading time. For example, no alarm may be generated if a user takes a reading at 8:55 AM or earlier per design or user preference.

The memory 814 may store data regarding the user's blood pressure. For example, the memory 814 may store data regarding maximum or minimum acceptable blood pressure or related readings (for example, systolic, diastolic, or pulse readings). If a reading is outside of an acceptable range, the processor 818 may cause the user to receive feedback (for example, visual or audio feedback) to alert the user of a potential problem. If a reading is outside of an acceptable range, a new reminder time and/or window may be set by the processor 818. For example and building on the previous example, if a user takes his blood pressure at 10:30 AM and the reading is outside of the user's acceptable range, a new reminder time will be set for 6 hours later and the window will be 3 hours. The user should then take his blood pressure with the device 810 between 4:30 PM and 7:30 PM. If the user does not then take his blood pressure by or before 7:30 PM, then the processor 818 may cause an alert to be generated.

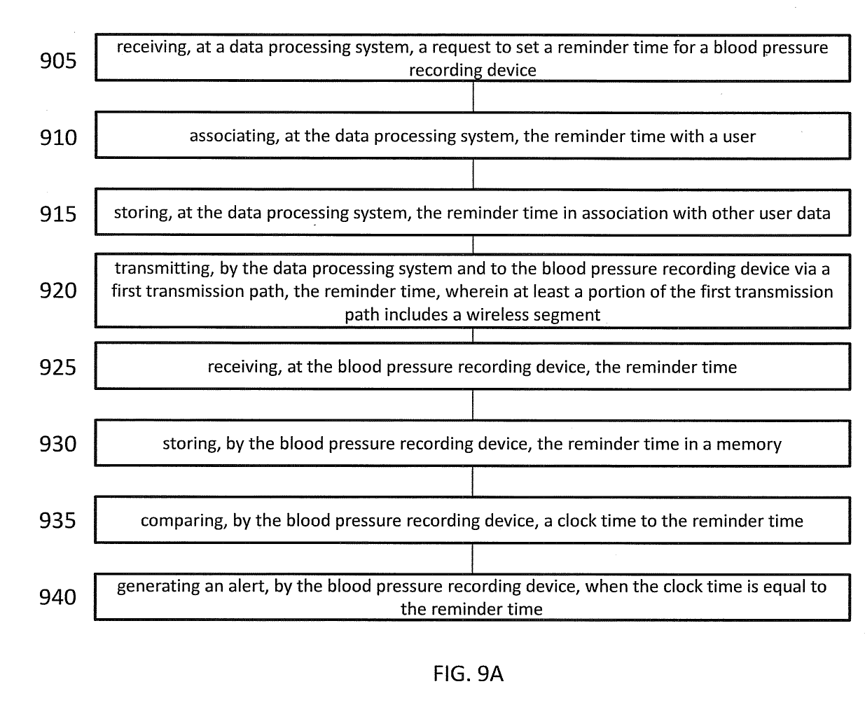

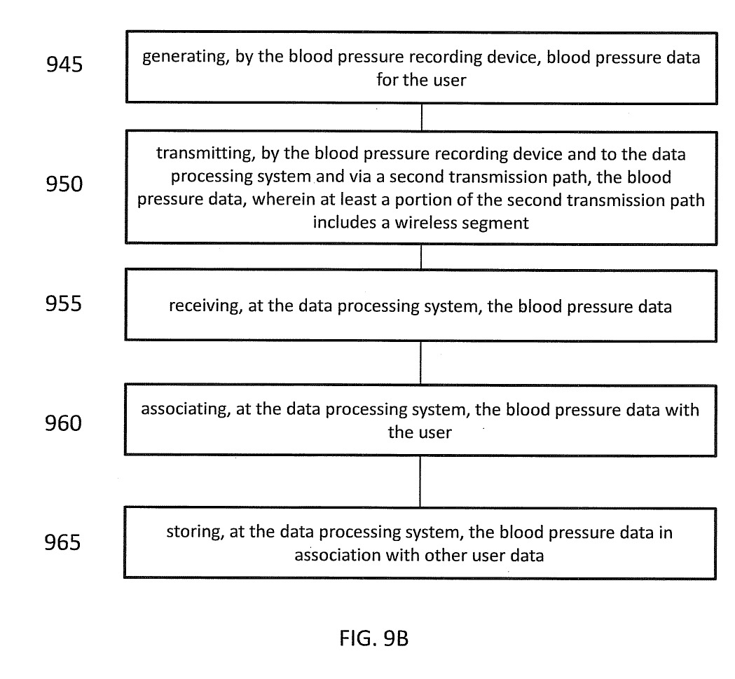

FIGS. 9A and 9B illustrate flow charts for methods of recording blood pressure of a user, according to certain inventive techniques. At step 905, a request to set a reminder time for a blood pressure recording device may be received by the data processing system. The request may be specified by a user through a portal. The reminder time may include or be associated with a window (discussed above) that may also be specified by a user. The user may specify multiple reminder times/windows such as multiple reminder times/windows for a given day of week or different reminder times/windows for different days of week. Other parameters may be specified by a user via a portal and be received by the data processing system, such as maximum/minimum blood pressure readings or pulse.

At step 910, the reminder time (and/or other data) may be associated with a user by the data processing system. At step 915, the reminder time (and/or other data) may be stored in association with other user data at the data processing system. At step 920, the reminder time (and/or other data) may be transmitted from the data processing system to the blood pressure recording device via a transmission path that includes a wireless segment.

At step 925, the reminder time (and/or other data) may be received at the blood pressure recording device. At step 930, the reminder time (and/or other data) may be stored in a memory by the blood pressure recording device. At step 935, a clock time may be compared to the reminder time by the blood pressure recording device. At step 940, an alert may be generated by the blood pressure recording device when the clock time is equal to the reminder time. Alternatively, an alert may be generated if a blood pressure reading is not taken within a window of time after the reminder time.

At step 945, blood pressure data for the user may be generated by the blood pressure recording device. At step 950, the blood pressure data may be transmitted to the data processing system via a transmission path that includes a wireless segment. At step 955, the data processing system may associate the blood pressure data with the user. At step 965, the data processing system may store the blood pressure data in association with other user data.

The methods depicted by the flowcharts in FIGS. 9A and 9B may also be understood in light of the following example. A user may use a computer to log into the data processing system via a portal. The user may be registered at the data processing system and may enter a reminder time and a window of time for taking his blood pressure. The user may also enter maximum and minimum blood pressure and pulse tolerances.

The data processing system may associate this data with the user and store this data in association with other data of the user (for example, other reminder times, historical blood pressure data, the user's biographical data, or the like). The data processing system may transmit the reminder time, window, and tolerances to the blood pressure recording device (for example, like device 810 illustrated in FIG. 8). This data may be transmitted from the data processing system to a wireless router over the Internet. This data may then be transmitted wirelessly from the router to the blood pressure recording device. Multiple reminder times/windows may be transmitted (for example, different times for different days of week). The blood pressure recording device may receive the data and store it in memory. The reminder time may then be compared to a clock time and an alert (for example, an audible or visual alert) may be generated. The user then knows it is time to take his blood pressure. Alternatively, the device may generate an alert if blood pressure has not been taken within the specified window of time.

The user then uses the blood pressure recording device to take his blood pressure and pulse. The blood pressure recording device then generates blood pressure and associated data including systolic data, diastolic data, and pulse. The recording device also includes a time stamp with this data. The data along with identifying information (for example, device serial number or user identification or credentials) is then wirelessly transmitted from the blood pressure recording device to the wireless router. The data and identifying information is then transmitted from the wireless router to the data processing system via the Internet. The data processing system associates the blood pressure data with the user according to the identifying information and stores the data in association with other user data (for example, previously taken blood pressure data, reminder times, the user's biographical data, or the like).

If the user's blood pressure or pulse exceeds the given tolerances, then the user may be required to retake his blood pressure/pulse before a subsequent time or during a subsequent window.

It will be understood by those skilled in the art that various changes may be made and equivalents may be substituted without departing from the scope of the novel techniques disclosed in this application. It is to be understood that the phraseology or terminology employed herein is for the purpose of description and not of limitation. In addition, many modifications may be made to adapt a particular situation or material to the teachings of the novel techniques without departing from its scope. Therefore, it is intended that the novel techniques not be limited to the particular techniques disclosed, but that they will include all techniques falling within the scope of the appended claims.

1. A method comprising:
receiving, at a data processing system, a request to set a reminder time for a blood pressure recording device;
associating, at the data processing system, the reminder time with a user;
storing, at the data processing system, the reminder time in association with other user data;
transmitting, by the data processing system and to the blood pressure recording device via a first transmission path, the reminder time, wherein at least a portion of the first transmission path includes a wireless segment;
receiving, at the blood pressure recording device, the reminder time;
storing, by the blood pressure recording device, the reminder time in a memory;
comparing, by the blood pressure recording device, a clock time to the reminder time; and
generating an alert, by the blood pressure recording device, when the clock time is equal to the reminder time.
